(19) United States
(12) Patent Application Publication
(10) Pub. No.: US 2003/0204096 A1
(43) Pub. Date: Oct. 30, 2003

(54) Enantioselective synthesis of azetidinone intermediate compounds

(75) Inventors: Xiaoyong Fu, Edison, NJ (US); Timothy L. Mc Allister, Westfield, NJ (US); T.K. Thiruvengadam, Kendall Park, NJ (US); Chou-Hong Tann, Berkeley Heights, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(73) Assignee: Schering Corporation

(21) Appl. No.: 10/441,391

(22) Filed: May 20, 2003

Related U.S. Application Data
(62) Division of application No. 10/105,710, filed on Mar. 25, 2002.

(51) Int. Cl. C07D 263/20

(52) U.S. Cl. 548/229

(57) ABSTRACT

The application relates to a process for preparing a compound of Formula I

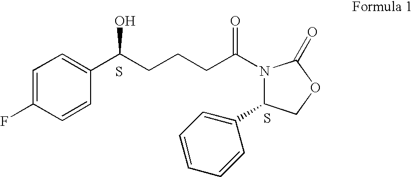

This compound is an intermediate used to produce compounds that are useful as hypocholesterolemic agents in the treatment and prevention of atheroschlerosis.

This application is a divisional of Ser. No. 10/105,710 filed Mar. 25, 2002 and claims priority from pending U.S. Provisional Application Serial No. 60/279,288 filed on Mar. 28, 2001.

### BACKGROUND OF THE INVENTION

This invention relates to a process for producing intermediates for hydroxy-alkyl substituted azetidinones. Hydroxy-alkyl substituted azetidinones, for example, 1-(4-fluorophenyl)-3(R)-[3(S)-hydroxy-3-(4-fluorophenyl)propyl)]-4(S)-(4-hydroxyphenyl)-2-azetidinone, are described in U.S. Pat. No. 5,767,115. These compounds are useful as hypocholesterolemic agents in the treatment and prevention of atheroschlerosis.

Processes for preparing the corresponding azetidinone without the 3-hydroxy substituent are claimed in U.S. Pat. Nos. 5,728,827 and 5,561,227. Other processes for preparing 1-(4-fluorophenyl)-3(R)-[3(S)-hydroxy-3-(4-fluorophenyl)-propyl)]-4(S)-(4-hydroxyphenyl)-2-azetidinone are disclosed in U.S. Pat. No. 5,631,365, U.S. Pat. No. 5,739,321 and U.S. Pat. No. 6,207,822 B1 (the '822 patent).

As per the procedure described in the '822 patent, the intermediate compound of Formula I, is protected with a suitable hydroxy-protecting group, such as a silyl protecting group such as that derived from chlorotrimethylsilane (TMSCI) or t-butyldimethyl-silyl chloride (TBDMSCI). This silylated product is further reacted with a silyl-enol ether silylating agent such as bistrimethylsilyl acetamide (BSA). A cyclizing agent such as a quaternary alkyl-, aryl-alkyl or arylalkyl-alkylammonium fluoride salt is then added to cause an intra-molecular cyclization of the previously silylated compound of Formula I. Finally, the protecting groups are removed from the cyclizied compound using conventional methods, such as treatment with a dilute acid, in order to form the hypocholesterolemic azetidinone having the Formula

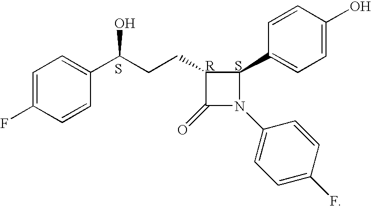

### SUMMARY OF THE INVENTION

This invention provides an improved, simple, high yielding process for preparing an intermediate compound useful in the production of azetidinones. The intermediate, a compound of Formula I:

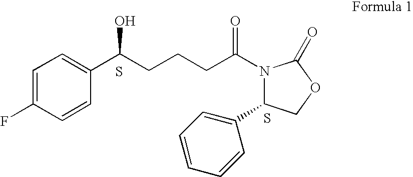

is prepared by a process which comprises:

a) mixing a compound of Formula II

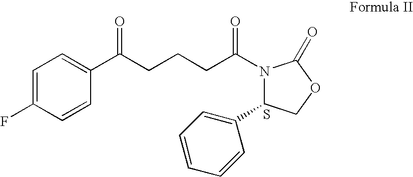

in tetrahydrofuran in the presence of an acid, or alternatively in tetrahydrofuran in the absence of an acid, to form a mixture;

b) combining the mixture of step a) with a catalyst selected from either (A) a compound selected from the group of compounds represented by Formula II, or (B) a compound of Formula IV,

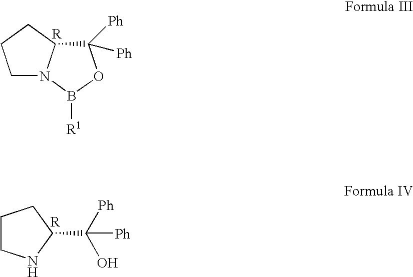

wherein R¹ of Formula III is a (C₁-C₆)alkyl and wherein R and S indicate stereochemistry at the chiral carbons;

c) reducing the ketone adjacent to the p-fluorophenyl with a borane-tetrahydrofuran complex; and

d) quenching the reaction with MeOH.

### DETAILED DESCRIPTION

In one embodiment, there is described herein a process for preparing a compound of Formula I

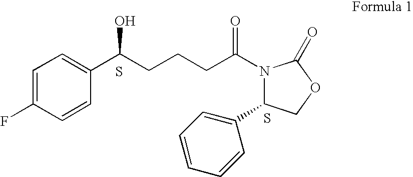

which comprises the steps (a)-(d) shown above.

In a preferred embodiment, the process comprises:

a) mixing a compound of Formula II in tetrahydrofuran in the presence of an acid to form a mixture;

b) combining the mixture of step a) with a catalyst selected from either (A) a compound selected from the group of compounds represented by Formula II, or (B) a compound of Formula IV

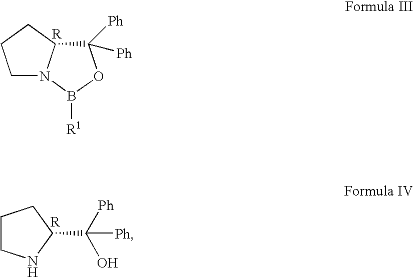

wherein R¹ of Formula III is a (C₁-C₆)alkyl and wherein R and S indicate stereochemistry at the chiral carbons;

c) reducing the ketone adjacent to the p-fluorophenyl with a borane-tetrahydrofuran complex; and

d) quenching the reaction with MeOH.

Except where stated otherwise, the following definitions apply throughout the present specification and claims. These definitions apply regardless of whether a term is used by itself or in combination with other terms. Hence the definition of “alkyl” applies to “alkyl” as well as to the “alkyl” portions of “alkoxy”, “alkylamino” etc.

“Alkyl” represents a straight or branched saturated hydrocarbon chain having the designated number of carbon atoms. Where the number of carbon atoms is not specified, 1 to 6 carbons are intended.

The acid in step a) is selected from the group consisting of BF₃.Et₂, BCl₃, p-toluene sulfonic acid, trifluoroacetic acid, methanesulfonic acid and camphorsulfonic acid.

If the catalyst of Formula IV is employed, it must be used in the presence of a trialkyl borate, preferably a trimethyl borate.

In another embodiment of the present invention, the ratio of the acid to the compound of Formula II is in a mole % of 1-10%, preferably 1-5%, more preferably in a mole % of 2-3%.

In another embodiment of the present invention, the ratio of the catalyst to the compound of Formula II of step b) is in a mole percent of 0.1-10%, preferably 1-5%, more preferably in a mole % of 2-3%.

In further embodiments of the present invention, the temperature of the reduction step c) is generally between −15 and 65° C., preferably between −10 and 55° C., more preferably between 0° and 30° C. and typically between 23° and 28° C.

In another embodiment of the invention, there is described a process for preparing a compound of Formula I

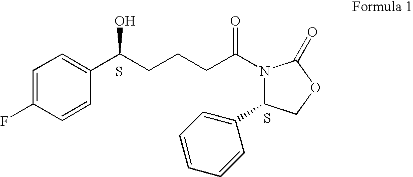

which process has no acid in step (a). The process, thus, comprises:

a) dissolving a compound of Formula II in tetrahydrofuran to form a mixture;

b) combining the mixture of step a) with a catalyst selected from either (A) a compound selected from the group of compounds represented by Formula III, or (B) a compound of Formula IV

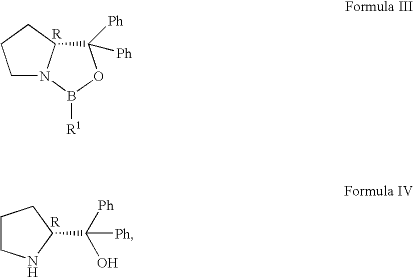

wherein R¹ of Formula III is a (C₁-C₆)alkyl and wherein R and S indicate stereochemistry at the chiral carbons;

c) reducing the ketone adjacent to the p-fluorophenyl with a borane-tetrahydrofuran complex; and

d) quenching the reaction with MeOH.

In a preferred embodiment of the alternate process (with no acid in step (a)) described immediately above, the temperature of the reduction step c) is between 23 and 28° C.

In another embodiment of the alternate process (with no acid in step (a)) described immediately above, the ratio of the catalyst to the compound of Formula II of step b) is in a mole % of 0.1-10%, preferably 1-5%, more preferably in a mole % of 2-3%.

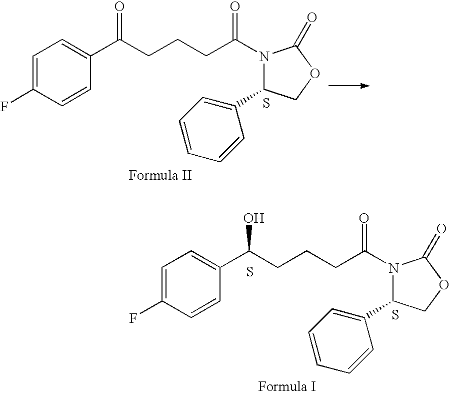

The present invention discloses a novel chemo selective and stereo selective reduction of the ketone adjacent to the p-fluorophenyl using a BH₃-THF complex. In a previous process patent, U.S. Pat. No. 6,207,822 B1 (the '822 patent), the disclosure of which is incorporated herein by reference thereto, there is disclosed a reduction of said ketone using BH₃ Me₂S (BMS) complex as a reducing agent. However, use of said BMS complex may lead to environmental concerns. The replacement of BMS with borane tetrahydrofuran complex eliminates the environmental issues raised by use of the BMS complex.

However, simple replacement of BH₃ Me₂S with BH₃-THF in the reduction generated a substantial amount of over-reduction of the amide bond, compared to the reduction of the ketone adjacent to the p-fluororophenyl, thus resulting in poor selectivity. Thus, initial experiments with BH₃-THF yielded a desirable % of desired enantiomer (SS) to the undesired enantiomer (SR), however, the solution yield was not optimized due to the production of the above-noted over-reduced by-product from the amide. Applicants found, in the present process, that reversing the addition sequence surprisingly overcame the poor chemoselectivity in the reduction. The production of the over-reduced by-product from the amide was significantly reduced while at the same time resulting in high diasteroselectivity in the product.

The new process calls for adding BH₃-THF to the solution of Formula II and (R)-tetrahydro-1-methyl-3,3-diphenyl-1H,3H-pyrrolo[1,2-c][1,3,2]oxazaborolidine (abbreviated as (R)-MeCBS) catalyst in THF (from Sigma-Aldrich, St. Louis, Mo.). Several experiments yielded results where the over-reduced by-product was minimized to <1% with diastereoselectivity of 97:3. In fact, the molar equivalent (eq) of BH₃-THF was kept to ˜0.6 eq, while % molar yields were generally over 97%. Similar results could be obtained with a “in-situ” prepared catalyst using the compound of Formula IV (R-diphenylprolinol) and trimethylborate. (See reference: M. Masui, T. Shioiri, Synlett, 1997, 273).

The following examples used to prepare the compound of Formula I illustrate the present invention, although such examples should not be construed as limiting the scope of the invention. Alternative reagents and analogous processes within the scope of the invention will be apparent to those skilled in the art. The product solutions of the following examples (which contain the compound of Formula I) can be directly used as such in subsequent process steps to make hydroxy-alkyl substituted azetidinones, or in the alternative, the compounds of Formula I can be crystallized or isolated using methods known and recognized by one of ordinary skill in the art.

### EXAMPLES

Abbreviations which are used in the description of the schemes, preparations and the examples are:

(R)—MeCBS=(R)-tetrahydro-1-methyl-3,3-diphenyl-1H,3H-pyrrolo[1,2-c][1,3,2]oxazaborolidine

THF=tetrahydrofuran

HPLC=High Performance Liquid Chromatography

MeOH=methanol

Atm=atmospheres

mL=milliliters

g=grams

PTSA=p-toluene sulfonic acid

CSA=(1S)-(+)-10-camphorsulfonic acid

TFA=trifluoroacetic acid

de=difference between SS % and SR %

### EXAMPLE 1

(Acid Absent in Step (a))

Fifty (50) g of the compound of Formula II was charged into a 1000 mL three necked round bottom flask equipped with a thermometer, N₂ inlet and addition funnel. 500 mL THF was charged to dissolve the 50 g of the compound Formula II at about 20° to 25° C. The batch was concentrated at 1 atm to a batch volume of about 150 mL. The temperature was adjusted to about 20° to 25° C. 4.2 mL of lab pre-formed (R)—MeCBS catalyst in toluene (3 mole %) was charged. 70.4 mL of 1 M borane THF complex in THF solution (from Aldrich Chemical Company, Milwaukee, Wis.) was slowly charged over 1.5 hrs at temperature between about 23° and 28° C. The batch was sampled for HPLC to monitor the progress of the reaction. After the reaction was judged complete, 20 mL of MeOH was slowly charged to keep the temperature below 25° C. in order to quench the reaction. The batch was concentrated under vacuum to afford a batch volume of about 100 mL at a temperature below 40° C. 250 mL of toluene and a solution of 5 mL sulfuric acid in 100 mL water was charged. The mixture was agitated for about 10 min. and the batch was allowed to settle. The bottom acid layer was split off. 100 mL of water was charged to wash the batch twice.

The batch was concentrated under vacuum at below 50° C. to afford a volume of about 100 mL. Results varied, but in general, yields of ˜99% and 95% de were obtained.

### EXAMPLE 2

(Acid (pTSA) Present in Step (a))

Fifty (50) kg of the compound of Formula II and 0.8 kg of p-toluene sulfonic acid (PTSA) was charged into a 300 gallon glass lined reactor equipped with a thermocouple, N₂ inlet and feed tank. 267 kg of dry THF was charged to dissolve the 50 kg of the compound Formula II and the p-toluene sulfonic acid at about 20 to 25° C. The batch was concentrated at 1 atm to a batch volume of about 185 liters. The temperature was adjusted to about 20 to 25° C. 200 liters of THF was charged to the batch. The batch was concentrated at 1 atm to a batch volume of about 185 liters. The temperature was adjusted to about 20 to 25° C. 3.4 kg of pre-formed (R)—MeCBS catalyst in toluene (3 mole %) was charged. 70.3 kg 1 M of borane THF complex in THF solution was slowly charged over 1.5 hours at a temperature range between about 23 and 28° C. The batch was sampled for HPLC to monitor the progress of the reaction. After the reaction was judged complete, using the same subsequent procedure as described in Example 1 (i.e. quenching with MeOH, vacuum concentration of the batch, etc., but in appropriate ratios of reagents for this example), the compound of Formula I was obtained in an average yield of 98.4%. A percentage yield of ˜97%, a solution yield of 100% and de of 93.6% was obtained.

### EXAMPLE 3

(Acid Present in Step (a))

Fifteen (15) kg of the compound of Formula II was charged into a 50 gallon glass lined reactor equipped with a thermocouple, N₂ inlet and feed tank. 150 liters of dry THF was charged to dissolve the 15 kg of the compound Formula II at about 20 to 25° C. The batch was concentrated at 1 atm to a batch volume of about 55 liters. The temperature was adjusted to about 20 to 25° C. 1.5 kg of preformed (R)—MeCBS catalyst in toluene (3 mole %) was charged. 18.55 kg of 1 M borane THF complex in THF solution was charged over 1.5 hours at a temperature range between about 23 and 28° C. The batch was sampled for HPLC to monitor the progress of the reaction. After the reaction was judged complete, using the same subsequent procedure as described in Example 1 (i.e. quenching with MeOH, vacuum concentration of the batch, etc., but in appropriate ratios of reagents for this example), the compound of Formula I was obtained in a yield of 100% with a de of 95.4%.

### EXAMPLE 4

Acid (CSA) Present in Step (a))

Thirty (30) g of the compound of Formula II and 0.386 g (2 mole %) of (1S)-(+)-10-camphorsulfonic acid (CSA) was charged in a 500 mL 3 necked round bottom flask equipped with a thermometer, N₂ inlet and addition funnel. 111 mL of dry THF was charged to dissolve the 30 g of the compound Formula II, and the (1 S)-(+)-10-camphorsulfonic acid at about 20 to 25° C. 2.2 mL of pre formed (R)—MCBS catalyst in toluene (3 mole %) was charged. 39.9 mL of 1 M borane THF complex in THF solution was slowly charged over 1.5 hours at a temperature range between about 23 and 28° C. The batch was sampled for HPLC to monitor the progress of the reaction. After the reaction was judged complete, using the same subsequent procedure as described in Example 1 (i.e. quenching with MeOH, vacuum concentration of the batch, etc., but in appropriate ratios of reagents for this example), the compound of Formula I was obtained. Results varied, but in general, ˜99% yield and ˜94% de were obtained.

### EXAMPLE 5

Using the method described above in example 4, other acids were substituted for CSA. This group of other acids included BF₃.OEt₂, BCl₃, trifluoroacetic acid (TFA) or methansulfonic acid. Results varied, but in general, all yielded results with favorable SS:RS ratios of ˜95-97% to ˜3-5% and a % de range from ˜91 to ˜93.8%.

In general, chemical yields close to 97% and over were obtained.

We claim:

1. A process for preparing a compound of Formula I 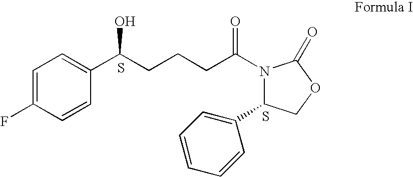
is prepared by a process which comprises:
a) mixing a compound of Formula II 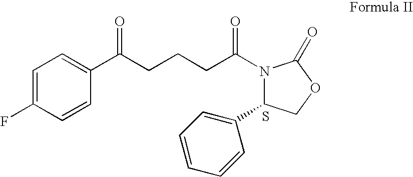
in tetrahydrofuran in the presence of an acid, or alternatively in tetrahydrofuran in the absence of an acid, to form a mixture;
b) combining the mixture of step a) with a catalyst selected from either (A) a compound selected from the group of compounds represented by Formula III, or (B) a compound of Formula IV, 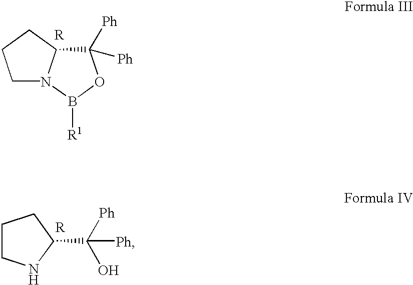
wherein R¹ of Formula III is a (C₁-C₆)alkyl and wherein R and S indicate stereochemistry at the chiral carbons;
c) reducing the ketone adjacent to the p-fluorophenyl with a borane-tetrahydrofuran complex; and
d) quenching the reaction with MeOH.

2. The process of claim 1 wherein the acid in step a) is BF₃.OEt₂, BCl₃, p-toluene sulfonic acid, trifluoroacetic acid, methanesulfonic acid, or camphorsulfonic acid.

3. The process of claim 2 wherein the catalyst of Formula IV is employed in the presence of a trialkyl borate.

4. The process of claim 3 wherein said trialkyl borate is a trimethyl borate.

5. The process of claim 2 wherein said acid is present in a 1-10 mole percent ratio with respect to said compound of Formula II.

6. The process of claim 2 wherein said acid is present in a 1-5 mole percent ratio with respect to said compound of Formula II.

7. The process of claim 2 wherein said acid is present in a 2-3 mole percent ratio with respect to said compound of Formula II.

8. The process of claim 1 wherein said catalyst is present in a 0.1-10 mole percent ratio with respect to said compound of Formula II.

9. The process of claim 1 wherein said catalyst is present in a 1-5 mole percent ratio with respect to said compound of Formula II.

10. The process of claim 1 wherein said catalyst is present in a 2-3 mole percent ratio with respect to said compound of Formula II.

11. The process of claim 1 wherein the temperature in the reduction step c) is between −15 and 65° C.

12. The process of claim 1 wherein the temperature in the reduction step c) is between −10 and 55° C.

13. The process of claim 1 wherein the temperature in the reduction step c) is between 0 and 30° C.

14. The process of claim 1 wherein the temperature in the reduction step c) is between 23 and 28° C.

15. The process of claim 1, wherein acid is present in step (a).

16. The process of claim 1, wherein acid is absent in step (a).

17. The process of claim 16 wherein the temperature in the reduction step c) is between 23 and 28° C.

18. The process of claim 16 wherein said catalyst is present in a 0.1-10 mole percent ratio with respect to said compound of Formula II.

19. The process of claim 16 wherein said catalyst is present in a 1-5 mole percent ratio with respect to said compound of Formula II.

20. The process of claim 16 wherein said catalyst is present in a 2-3 mole percent ratio with respect to said compound of Formula II.

21. A compound of Formula I 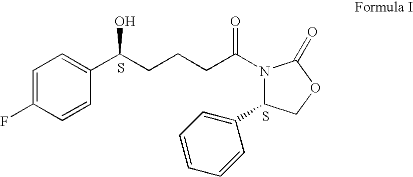
formed by the process of claim 15.

22. A compound of Formula I 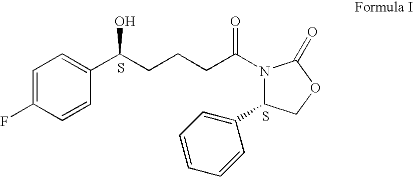
formed by the process of claim 16.
